(19) United States
(12) Patent Application Publication
(10) Pub. No.: US 2009/0060842 A1
(43) Pub. Date: Mar. 5, 2009

(54) METHOD FOR DIAGNOSIS AND TREATMENT OF PROSTATITIS

(76) Inventors: Jens Fehre, Hausen (DE); Ralf Nanke, Neunkirchen am Brand (DE); Martin Stetter, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 12/203,262

(22) Filed: Sep. 3, 2008

(30) Foreign Application Priority Data
Sep. 3, 2007 (DE) 10 2007 041 837.1

(51) Int. Cl. A61K 49/06; A61P 35/00

(52) U.S. Cl. 424/9.3; 514/789

(57) ABSTRACT

In a method for diagnosis and treatment of a patient with regard to prostatitis, a differential diagnosis of prostatitis versus prostate cancer and/or BPH is conducted on a patient using a cost-effective diagnosis method. Given diagnosed prostatitis, a cost-effective decision test is implemented on the patient to determine whether the patient exhibits complaints locally in the region of the prostate or in the rest of the body. A molecular inflammation marker is administered to the patient, wherein a cost-effective imaging method is implemented in the region of the prostate of the patient given local complaints or a cost-intensive imaging method is implemented on the entire patient given complaints in the rest of the body. An inflammation-inhibiting treatment is implemented in the body region of the patient that is detected as inflamed via the respective imaging method.

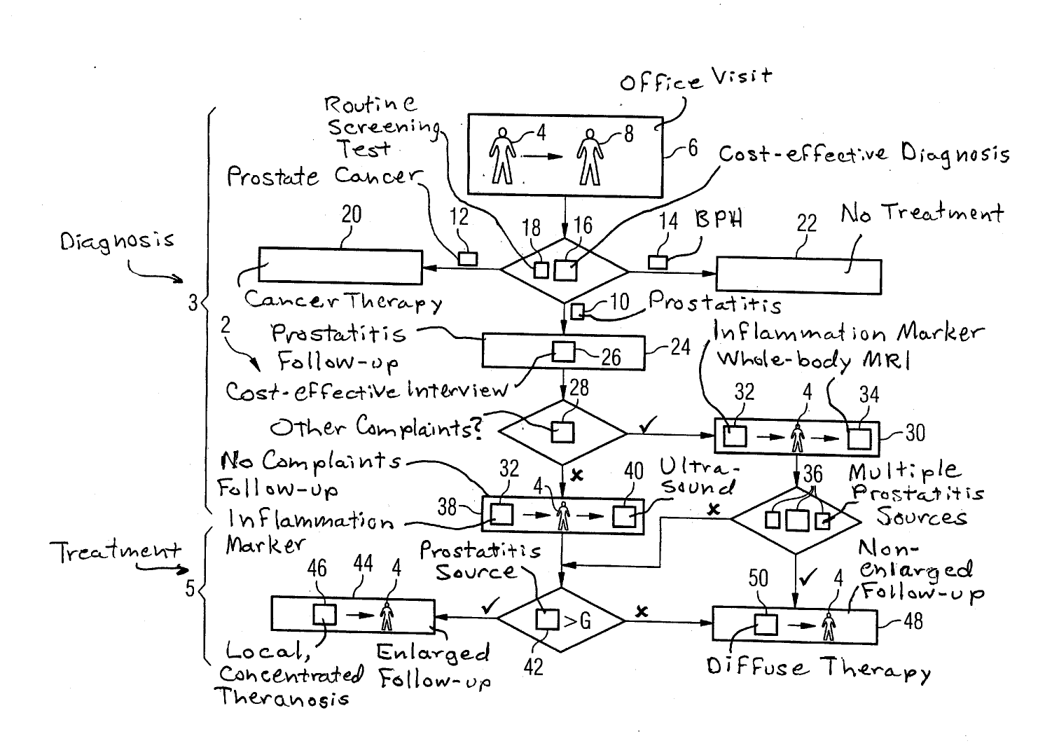

### BACKGROUND OF THE INVENTION

1. Field of the Invention

The present invention concerns a method for diagnosis and treatment of a patient with regard to prostatitis.

2. Description of the Prior Art

The diagnosis and treatment of prostate inflammation or bacterial prostatitis (or prostatitis for short) is severely limited by severe weaknesses of the presently used methods in terms of their efficiency. Clinically established methods today can only imprecisely diagnose prostatitis and distinguish it from prostate cancer. Many incorrect treatments result form this since prostatitis is incorrectly diagnosed as cancer and treated similarly. Failure to implement treatments likewise result since existing prostatitis is not diagnosed and thus is not treated. In the treatment of correctly diagnosed prostatitis, existing pharmaceutical inflammation treatments work non-specifically in the entire body, and can cause significant side effects.

The clinically established workflow today for diagnosis and treatment of prostatitis is to implement a palpation, an ultrasound or a urine examination. For prostatitis, a search is made for inflammatory bacteria. The PSA test (namely a blood test) in which a cancer antigen is tested relative to a threshold is known for prostate cancer. If the value is beyond the threshold, a biopsy is conducted on the patient. The biopsy result may be uncertain since the cancer source can be very small, and cannot be shown by imaging, and thus must be hit more or less at random in the biopsy.

Today antibiotics are administered in the case acute prostatitis (which is established by palpation). In the case of chronic prostatitis, which may only possibly be found by palpation finding or for which calcium residues are only possibly visible in ultrasound, a long-term therapy with special antibiotics results. A prostate abscess as a result of prostatitis, which today is for the most part determined by palpation, is typically operatively treated.

These disadvantages lead to a sub-optimal care of a patient with regard to diagnosis and treatment of prostatitis.

Novel screening, diagnosis and treatment methods presently exist or are in development that exhibit greater diagnostic precision and diagnostic impact based on molecular mechanisms.

For example, a more cost-effective molecular screening test for differential diagnosis of prostatitis versus prostate cancer and a benign prostate enlargement (BPH—benign prostate hyperplasia) is described in the German Patent Application 10 2007 028 659.9 filed on 21 Jun. 2007, for example. This test can do without elaborate or expensive imaging like MR and is based on infrared technology (NIR—near infrared). Molecular markers that selectively accumulate in prostate or prostatitis tissue thereby play a decisive role.

Furthermore, a method for magnetic resonance imaging with ferromagnetic, molecularly marked particles as a contrast agent is described in United States patent application filed simultaneously herewith Attorney Docket No. P08,0241, with the same priority as the present application. The particles specifically accumulate in prostate cancer tissue and thus enable a better representation.

Moreover, a molecular, contrast-enhanced ultrasound examination of the prostate is described in United States patent application filed simultaneously herewith Atty. Docket No. P08,0239, with the same priority as the present application. Prostate cancer can be distinguished from healthy tissue in an ultrasound image with the use of targeted microbubbles that selectively attach to cancer tissue.

A method for ultrasound theranosis is also described in United States patent application filed simultaneously herewith Atty. Docket No. 08,0245, with the same priority as the present application. Molecular markers and pharmaceutical transporters are hereby simultaneously administered. The markers indicate the extent of the prostatitis; the therapeutic agent is only released at the inflammation site via local ultrasound triggering.

Furthermore, a method that allows a selective medicinal treatment of inflammations (but also of prostatitis) on a molecular basis is described in German Patent Application 10 2007 028 661.0 filed on 21 Jun. 2007.

These three methods can analogously also be applied for the case of prostatitis in the event that a corresponding prostatitis or, respectively, inflammation marker is used instead of the described marker.

The cited methods have various properties with regard to precision, diagnostic bandwidth, therapeutic impact and costs. A perfect care of every patient would be ensured via the application of all cited novel methods with each patient. However, this is unrealistic due to the health care cost explosion that would be triggered by this.

### SUMMARY OF THE INVENTION

An object of the present invention is to provide an improved method for diagnosis and treatment of a patient with regard to prostatitis.

The invention achieves optimal care of a patient with minimal costs by a medical workflow defined for prostatitis, in which workflow the novel (for example molecularly supported) diagnosis and treatment techniques described above are used optimally for each patient with regard to their effectiveness, and at the same time unnecessary and inefficient steps are avoided.

The object is achieved by a method for diagnosis and treatment of a patient with regard to prostatitis, with the following steps:
a differential diagnosis of prostatitis versus prostate cancer and/or BPH is conducted on a patient using a cost-effective diagnosis method,
given diagnosed prostatitis, using a cost-effective decision test on the patient it is determined whether said patient exhibits these complaints locally in the region of the prostate or also in the remainder of the body,
a molecular inflammation marker is then administered to the patient, wherein—according to the decision test—a cost-effective imaging method is implemented only locally in the region of the prostate of the patient given local complaints of the patient, or a cost-intensive imaging method is implemented on the entire patient given complaints in the rest of the body,
an inflammation-inhibiting treatment is implemented in the body region of the patient that is detected as inflamed via the respective imaging method.

The care process on a patient begins with complaints or, respectively, conspicuities of the prostate. These conspicuities can, for example, exist in an increased PSA level that will be examined further using a palpation, a transrectal ultrasound examination and/or biopsy with subsequent histology. The goal of this classical diagnosis is the finding of prostatitis versus prostate cancer or BPH. All of these measures respectively represent a cost-effective diagnosis method.

In the case of the diagnosis of prostate cancer, the method branches to a workflow for cancer treatment that should not be explained in detail here. If the assessment is without pathological findings, this means that no illness exists or BPH is present; the examination is ended or, respectively, the patient can be treated further in an uncomplicated manner, which is likewise not explained in detail here.

Given diagnosed prostatitis, the decision test ensues with the goal of clarifying whether complaints exist only in the region of the prostate or also in other bodies of the patient. It should be noted that a chronic prostatitis does not have to entail acute complaints.

Due to the molecular inflammation marker, the inflammation can consequently actually be graphically detected. If the patient has no complaints or only has complaints in the region of the prostate, a local examination of the prostate can be proceeded with that is more cost-effective than an imaging method of the entire body of the patient but is more precise than the aforementioned, cost-effective diagnosis method. The position and the size of the inflammation source are thus detected. In the case of a prostate abscess, an operative treatment is often required. The information so determined can serve for OP planning. In the case of prostatitis, it can serve to decide whether an expensive, local and powerful therapy is necessary or a diffuse, cost-effective therapy is sufficient.

If complaints of the patient also exist outside of the prostate (since inflammations can propagate in the body), the detection of optimally all inflammation sources in the body is to be implemented with a suitable method. Such a method is relatively expensive and elaborate and, is therefore only used when information about the entire body are required. As a result, information is acquired about whether and to what extent inflammation sources exist in the body outside of the prostate and information about the size and position of a prostatitis source.

A therapy of the patient now follows an occurred diagnosis. According to the invention, this is implemented only in the region of the actual inflammation, and thus the patient is treated only there where it is necessary for the well-being of the patient.

Through the method according to the invention, a novel medical workflow is achieved that allows novel, molecularly supported screening, diagnosis and therapy methods to be used, such that the patient care is the best possible but unnecessary steps are thereby consistently omitted. The efficiency of the prostate care can thus be increased. Higher care quality results with simultaneously reduced costs, which is of particularly great importance in light of the demographic change and the fact that prostate complaints predominantly affect older men.

Due to the more precise coordination and higher impact of the treatment, treatment durations are minimized, recovery rates are increased and side effects are minimized.

Due to the better and more precise information of the patient, but also due to the omission of unnecessary diagnostic steps and the non-invasive nature of the diagnostic or therapeutic methods, the patients are exposed to fewer mental and other stresses.

Through the method according to the invention, the efficiency of the care for a given patient is maximized and at the same time the diagnosis or treatment costs are minimized.

A screening test can also be implemented as a diagnosis method. Abnormalities can thus be detected in the framework of broadly applied screening tests of the male elderly population. Due to the large number of tests, a particularly cost-effective method can be used in the screening that can draw a yes/no conclusion about the presence of prostatitis with high sensitivity and specificity, and in particular can differentiate this from prostate cancer and BPH.

A molecular contrast enhanced test can also be implemented as a diagnosis method. An example of such a method is, for example, a molecular contrast enhanced ultrasound examination or infrared examination as mentioned above. This test can also be used outside of a screening.

A patient interview, for example a conventional interview or, respectively, examination of the patient by a physician, can be conducted as a decision test.

An ultrasound examination with the molecular inflammation marker (thus a molecularly, contrast agent-enhanced ultrasound examination) can be conducted as a local imaging method in the region of the prostate.

By contrast, a whole-body imaging method (such as, for example, MRI) with molecular, inflammation-specific contrast enhancement is suitable as an imaging method for the entire patient, for example.

From the results of the diagnosis it can be differentiated whether the size of an inflamed prostatitis source lies above or below a predeterminable limit value. In the event of a size that lies above the limit value, a cost-intensive local treatment can then be implemented. In the case of a large or chronic prostatitis source, for example, an expensive, local and highly effective molecularly supported therapy is indicated.

Given a size of the prostatitis source below the limit value or given multiple inflammation sources (distributed in the body, for example), a cost-effective whole-body therapy (for example a diffuse treatment) can be conducted in the body of the patient. This can be a conventional antibiotic administration or a molecularly targeted active substance administration to all inflammations as mentioned above.

A theranostic ultrasound treatment with a molecular marker and/or a precisely targeted release of therapeutic agent can also be implemented as a treatment. For example, in a theranostic ultrasound approach a selective marker binding to inflamed tissue can be coupled both with a contrast agent and with a therapeutic agent. The contrast agent there shows the size of the source in the ultrasound examination and thus also the treatment course or, respectively, success. The therapeutic agent is simultaneously released in the diseased tissue with precise targeting. This ensues in that, for example, the therapeutic agent is enclosed in air bubbles or, respectively, microbubbles which are destroyed with ultrasound.

### BRIEF DESCRIPTION OF THE DRAWING

The single FIGURE is a flow chart of an embodiment for diagnosis and treatment of a prostatitis patient in accordance with the invention.

### DESCRIPTION OF THE PREFERRED EMBODIMENTS

The FIGURE shows a workflow 2 for prostatitis diagnosis 3 and treatment 5 in which, in an office visit 6, a patient 4 seeks out a physician 8 due to complaints or abnormalities of the prostate. The physician conducts a cost-effective (economical, low involvement of complicated medical equipment) diagnosis method 16 on the patient 4 for differential diagnosis of prostatitis 10 versus prostate cancer 12 or BPH 14.

Using a variant of the invention, the patient 4 seeks out the physician 8 not due to complaints but rather to participate in a routine screening test 18 that replaces the diagnosis method 16.

If the result of the diagnosis method 16 or of the screening test 18 is prostate cancer 12, a cancer therapy 20 (not explained in detail here) ensues. If the result is BPH 14 or no finding, a BPH treatment or no further treatment at all ensues in Step 22.

A cost-effective interview 26 or examination of the patient 4 by the physician 8 ensues in Step 24 in the event of prostatitis 10. This serves as a differentiation test. Namely, here it is differentiated or, respectively, determined whether the complaints of the patient occur only locally at the prostate or, respectively, whether he has any complaints at all, or whether the patient 4 also has other complaints 28 in the rest of the body.

An administration of an inflammation marker 32 now ensues in both following steps 30 or 38. In the case of other complaints 28, Step 30 branches and an expensive whole-body MRI 34 of the patient 4 is conducted. This serves to decide whether or not the patient exhibits multiple inflammation sources 36 in the entire body.

Alternatively, proceeding from Step 24 the workflow branches to Step 38 given no other complaints 28 at all, in which Step 38 inflammation markers 32 are likewise administered to the patient 4 as mentioned above; however an ultrasound 40 is conducted on the patient 4 in the region of the prostate.

The assessment of the size of the prostatitis source 42 relative to a limit value G ensues after Step 38 or in the event that multiple sources 36 were found in the patient after Step 30. If the source 42 is larger than the limit value G, a local and expensive but highly effective theranosis 46 ensues in Step 44, namely a theranostic ultrasound treatment with molecular markers and drug delivery.

If the prostatitis source 42 is smaller than the limit value G, or if multiple sources 36 were found in the patient 4 after Step 30, a diffuse and cost-effective therapy 50 ensues in Step 48 as a whole-body inflammation therapy of the patient 4.

Although further modifications and changes may be suggested by those skilled in the art, it is the intention of the inventors to embody within the patent warranted hereon all changes and modifications as reasonably and properly come within the scope of their contribution to the art.

We claim as our invention:

1. A method for diagnosis and treatment of a patient with regard to prostatitis, comprising the steps of:
implementing a differential diagnosis of prostatitis versus prostate cancer and/or BPH on a patient using a cost-effective diagnosis method;
given diagnosed prostatitis, using a cost-effective decision test on the patient to determine whether the patient exhibits complaints locally in the region of the prostate or in the rest of the body;
administering a molecular inflammation marker to the patient, wherein
a cost-effective imaging method is implemented in the region of the prostate of the patient given local complaints,
a cost-intensive imaging method is implemented on the entire patient given complaints in the rest of the body; and
an inflammation-inhibiting treatment is implemented in the body region of the patient that is detected as inflamed by the respective imaging method.

2. A method according to claim 1, comprising conducting a screening test as said diagnosis method.

3. A method according to claim 1 comprising conducting a molecularly contrast-enhanced test as said diagnosis method.

4. A method according to claim 1, comprising conducting a patient interview is implemented as said decision test.

5. A method according to claim 1, comprising conducting an ultrasound examination or an MRI with molecular inflammation markers as said imaging method.

6. A method according to claim 1, comprising:
implementing a cost-intensive local treatment as said treatment given a size of an inflamed prostatitis source above a predeterminable limit value, and implementing a cost-effective whole-body therapy given a size of the prostatitis source below the limit value or given multiple inflammation sources in the body of the patient.

7. A method according to claim 1, comprising implementing a theranostic ultrasound treatment with a molecular marker and/or a precisely targeted release of therapeutic agent as a local treatment.
